(19) United States
(12) Patent Application Publication
(10) Pub. No.: US 2025/0288645 A1
(43) Pub. Date: Sep. 18, 2025

(54) USE OF GsMTx4 AND GsMTx4 CONJUGATES TO IMPROVE RECOVERY FROM JOINT INJURY

(71) Applicants: University of Rochester, Rochester, NY (US); The Research Foundation for the State University of New York, Amherst, NY (US)

(72) Inventor: Whasil LEE

(73) Assignees: University of Rochester, Rochester, NY (US); The Research Foundation for the State University of New York, Amherst, NY (US)

(21) Appl. No.: 18/862,533

(22) PCT Filed: May 4, 2023
(86) PCT No.: PCT/US2023/021084

§ 371 Date: Nov. 3, 2024

(51) Int. Cl. A61K 38/17; A61K 9/00; A61K 47/54; A61K 47/66; A61P 19/02

(52) U.S. Cl. CPC A61K 38/1767; A61K 9/0019; A61K 47/557; A61K 47/66; A61K 47/665; A61P 19/02

(57) ABSTRACT

The invention provides methods and products to reduce one or more symptoms from injury to a synovial joint. The methods and products relate to injecting into or around cartilage of the synovial joint the peptide GsMTx4 or a composition targeting GsMTx4 to the cartilage. The synovial joint can be, for example, a knee joint, shoulder joint, hip joint, or a facet joint of the spine.

### CROSS-REFERENCE TO RELATED APPLICATIONS

This application claims priority to, and the benefit of, U.S. Provisional Patent Application Nos. 63/338,434, filed May 4, 2022, and 63/458,903, filed Apr. 12, 2023. The contents of both applications are hereby incorporated herein by reference in their entirety.

### STATEMENT OF FEDERAL FUNDING

This invention was made with government support under R35 GM147054, R01 AR082349, T32 AR076950, and P30 AR069655 awarded by the National Institutes of Health. The government has certain rights in the invention.

### PARTIES TO JOINT RESEARCH AGREEMENT

Not applicable.

### BACKGROUND OF THE INVENTION

Damage to load-bearing joints, whether from trauma or from the deterioration of cartilage and joint disfunction due to osteoarthritis (“OA”), is a common source of pain and reduced quality of life for many individuals. Some 300 million people globally are estimated to suffer from osteoarthritis, while rotator cuff tears are one of the most common orthopedic injuries and cause substantial pain and disability.

More than 32 million Americans and 300 million people globally suffer from OA, a debilitating disease whose hallmarks are progressive cartilage degeneration, joint pain, and decreased mobility. Several disease-modifying OA drugs (DMOADs) have been developed, yet none have demonstrated long-term efficacy and safety. Cartilage maintenance and degeneration are heavily influenced by mechanical stimuli via mechanotransduction of chondrocytes, the cell type responsible for cartilage matrix turnover and mechanical integrity.

In the United States, shoulder related pain results in some 4.5 million physician office visits annually. Rotator cuff pathology is the leading cause of shoulder-related disability. The rotator cuff can be injured from either acute trauma or as part of a progressive degenerative process. Asymptomatic degenerative rotator cuff tears (sometimes abbreviated as “RCT”), including partial- and full-thickness, are common in the general population with an estimated prevalence of 20% of individuals in their 60s and up to 80% of individuals in their 80s, and asymptomatic tears become symptomatic within 2-5 years in 30-40% of patients. Size and thickness or tears often guide treatment decisions as these factors relate to enlargement of the tear over time. Approximately 50% of full-thickness tears will enlarge over a 5-year time frame, and a combination of fatty infiltration into the cuff muscles and further tendon degeneration will substantially decrease rate of successful surgical intervention. Surgical treatment of massive rotator cuff tears, defined as full thickness tears >5 cm in the coronal plane or tears involving 2 or more tendons, remains a clinical challenge as there are high structural failure rates and poor clinical outcomes following repair or reconstruction.

It would be useful to have new interventions for reducing the pain and damage from traumatic injury to synovial joints such as the shoulder and the knee, and to thereby reduce the risk of developing OA in the affected joint. Surprisingly, the present invention fulfills these and other needs.

### BRIEF SUMMARY OF THE INVENTION

In a first group of embodiments, the invention provides methods of reducing one or more symptoms from a synovial joint injury in a subject in need thereof, said method comprising administering to said synovial joint an effective amount of GsMTx4 or of a targeted GsMTx4 composition that improves delivery of GsMTx4 to cartilage in said synovial joint. In some embodiments, what is administered to said synovial joint is GsMTx4. In some embodiments, the targeted GsMTx4 composition that improves delivery to said cartilage is a fusion peptide of GsMTx4 and WYRGRL (SEQ ID NO:1). In some embodiments, the targeted GsMTx4 composition that improves delivery to said cartilage is a fusion peptide of GsMTx4 and GGWYRGRL (SEQ ID NO:2). In some embodiments, the targeted GsMTx4 composition that improves delivery to said cartilage is biotin conjugated to said GsMTx4. In some embodiments, the targeted GsMTx4 composition that improves delivery to said cartilage is streptavidin conjugated or fused to said GsMTx4. In some embodiments, the administration is by injection into or around cartilage in said synovial joint. In some embodiments, the reducing of one or more symptoms is a reduction in pain. In some embodiments, the reducing of one or more symptoms is a reduction in cartilage degeneration. In some embodiments, the synovial joint is a knee joint. In some embodiments, the injury to said knee joint is a tear in an anterior cruciate ligament. In some embodiments, the reducing of one or more symptoms in said knee joint is a reduction in pain. In some embodiments, the reducing of one or more symptoms from said injury to said knee joint is a reduction of impairment of gait of said subject. In some embodiments, the synovial joint is a shoulder joint. In some embodiments, the reducing of one or more symptoms in said shoulder joint is a reduction in pain when said subject moves said joint. In some embodiments, the reducing of one or more symptoms from an injury to said shoulder joint is a reduction in impairment of movement of said shoulder joint. In some embodiments, the synovial joint is a facet joint of a spine. In some embodiments, the reducing of one or more symptoms in said facet joint is a reduction in pain when said subject moves said joint.

In a second group of embodiments, the invention provides products comprising a composition of GsMTx4 or of a targeted GsMTx4 composition that improves delivery of GsMTx4 to cartilage in a synovial joint, for use in reducing one or more symptoms from an injury to said synovial joint. In some embodiments, the synovial joint is a knee. In some embodiments, the injury to said synovial joint in said knee is an anterior cruciate ligament tear. In some embodiments, the reduction of said one or more symptoms from said injury to said knee is pain. In some embodiments, the reduction of said one or more symptoms from said injury to said knee is a reduction in impairment of gait consequent to said injury. In some embodiments, the synovial joint is a shoulder. In some embodiments, the injury to said shoulder is a rotator cuff tear. In some embodiments, the reduction of said one or more symptoms from said injury to said shoulder is pain. In some embodiments, the reduction of said one or more symptoms from said injury to said shoulder is a reduction in impairment of movement of said shoulder consequent to said injury. In some embodiments, the synovial joint is a facet joint of a spine. In some embodiments, the reduction of said one or more symptoms from said injury to said facet joint of said spine is a reduction of pain when said facet joint of said spine is moved. In some embodiments, the synovial joint is a hip joint. In some embodiments, the reduction of said one or more symptoms from said injury to said hip joint is pain.

### BRIEF DESCRIPTION OF THE FIGURES

FIG. 1A. FIG. 1A presents four photos of knee joints, two from control animals and two from animals that have been subjected to bilateral ACL injury (“bi-ACL-I”). For orientation, the bones and meniscus in the upper left photo are labeled as follows: F=femur, T=tibia, and M=meniscus. The bones and meniscus of the other three photos are in the same orientation as those in the upper right, although the tibias in the animals that have undergone bilateral ACL injury have been displaced relative to those of the uninjured controls. Referring to the top two photos, the left-hand photo of the joint of the uninjured control animal shows a thick band of cartilage overlaying the bone of both the femur and the tibia (the stained red band in the original photos shows as a medium-dark gray band in the black and white reproductions). In contrast, in the animal with the bilateral ACL injury, the cartilage has been eroded almost completely away from a substantial portion of the tibia, and is notably thinned on the femur. Referring to the bottom two photos, one can see substantial synovial inflammation (synovitis), as indicated by the increase in cells in the synovial membrane in the joint subjected to bilateral ACL injury compared to the joint from the uninjured control.

FIG. 1B. FIG. 1B presents graphs comparing the osteoarthritis severity, or “OARSI” score for the femur and for the tibia, respectively, as well as the synovitis score. The graphs for the femur and for the tibia show that the OARSI score (abbreviated “Sco” in FIG. 1B) is much higher for the bones from joints that have undergone bilateral ACL injury compared to those from controls, as is the synovitis score.

FIG. 1C. FIG. 1C presents photographs of the expression of Piezo1 on the cartilage on the surface of a femur (“F”) of a mouse whose ACL was not injured (top photo, labeled “CTL”), and of the expression of Piezo1 in the cartilage on the surface of the femur of a mouse four weeks after bilateral ACL injury (“bi-ACL-I”). As can be seen from the photos, the expression of Piezo1 is significantly upregulated in the cartilage of the joint that has been injured.

FIG. 1D. FIG. 1D, left side, presents a graph quantitating the expression level of Piezo1 in the cartilage of animals four weeks after bilateral ACL injury relative to the corresponding joint of control animals. On the right side, FIG. 1D presents a graph showing the relative resting levels of Ca²⁺ in the joint of animals four weeks after bilateral ACL injury relative to the corresponding joint of control animals.

FIG. 1E. FIG. 1E is a diagram showing impact loading applied to cartilage over a portion of a mouse bone as part of an assay for studying the resulting area of chondrocyte death.

FIG. 1F. FIG. 1F presents photos of a bone of a control mouse subjected to impact loading, and of a bone of a mouse with a bilateral ACL injury subjected to impact loading. Although it is harder to see in the black-and-white reproduction than in the color original, in which the dead cells are red and the living cells are green, the area of dead cells is much larger in the cartilage of the animal with the bilateral ACL injury than in cartilage of the mouse that was not first subjected to bilateral ACL injury.

FIG. 1G. FIG. 1G presents a graph quantifying the areas of cell death seen in control animals and in animals with a bilateral ACL injury one week (“1w”) after the animals who underwent bilateral ACL injury were injured (“post-I”), and four weeks (“4w”) after the animals who underwent bilateral ACL injury were injured.

FIG. 1H. FIG. 1H is a graph plotting, on the Y axis, the relative amount of Piezo1 in the cartilage of control animals (“CTL”) and animals that have undergone bilateral ACL injury (“bi-ACL”), as shown by binding of an anti-Piezo1 antibody, and, on the X axis, the area of injured (actually, dead) cells following the application of a punch force on the cartilage.

FIG. 2A. FIG. 2A presents two photos, both from animals that have undergone bilateral ACL injury. The animal in the photo shown on the left was injected after the injury with saline solution (“+saline”), while the one on the right was injected at the same time points post-injury with GsMTx4 (“+GsMTx4”). The cartilage on the bone of the mouse treated with saline is severely eroded and disrupted on the femur and the tibia, while there is still a layer of cartilage almost all of the femur and tibial surface in the animal treated with GsMTx4.

FIG. 2B. FIG. 2B is a graph plotting (on the Y axis) the combined OARSI score for the femur and the tibia of (from left to right across the X axis) animals that were not injured, animals with bilateral ACL injury (“bACL”), animals with bilateral ACL injury, followed by subcutaneous injection of saline solution, and animals with bilateral ACL injury, followed by subcutaneous injection of GsMTx4.

FIG. 2C. FIG. 2C presents two sets of two photos from animals that have undergone bilateral ACL injury. The animal in the photos in the top row was injected after the injury with saline solution (“ACLL+saline”), while the animal in the bottom row was injected at the same time points post-injury with GsMTx4 (“ACLI+GsMTx4”). The left photos show the amount of dead cells (in red, in the color original, compared to living cells, which have taken up a green label) and after application of a 1 mJ force. FIG. 2D presents a graph showing the results of the studies. The Y axis shows the area of dead cells per μm². The X axis shows both whether the animals are uninjured controls (“ACL-I−”) or have undergone bilateral ACL injury (“ACL-I+”), and whether they have been left untreated (“Admin-”) or have been treated with saline or with GsMTx4.

FIG. 3. FIG. 3 presents four graphs depicting aspects of the gait of mice three weeks after bilateral ACL injury (“ACLI”), followed by weekly subcutaneous injections of either saline or of GsMTx4, and of mice that were not subjected to an ACL injury but were injected with GsMTx4 at the same time points.

### DETAILED DESCRIPTION

Surprisingly, the present invention provides methods and compositions for reducing joint pain and damage, such as the mechanical stimuli-induced death of chondrocytes. As described in more detail below, joint injury is a risk factor in later developing osteoarthritis (“OA”). In some aspects, the invention relates to reducing joint damage and joint pain after a joint has undergone a traumatic injury. In some aspects, the inventive methods and compositions are expected to slow cartilage degeneration and reduce the development of severe osteoarthritis in the injured joint. In some aspects, the invention relates to reducing joint pain following a joint injury to load bearing joints or to synovial joints. In some aspects, the joint injury is to a knee ligament, such as the anterior cruciate ligament, or “ACL.” In some aspects, the invention relates to reducing damage, and consequent pain, due to shoulder injuries that cause rotator cuff tears.

“GsMTx4” and “GsMTx-4” refer to a 34-amino acid peptide (SEQ ID NO:4) originally isolated from the venom of a South American tarantula, Grammostola spatulata. The peptide blocks cation-selective stretch-activated channels.

Surprisingly, studies in animal models have revealed that GsMTx4 administered after injury to a joint, such as the knee, results in reduced cartilage degeneration, decreased mechanical-impact-induced chondrocyte death, and decreased joint-innervating nerves with pain-biomarkers, compared to animals administered saline solution post-injury instead of GsMTx4 GsMTx4. Animals injured in the knee and then administered GsMTx4 exhibited notably better gait than animals administered saline. The better gait of animals administered GsMTx4 compared to those administered saline is believed to mean that the animals felt less pain following GsMTx4 treatment compared to those administered to which saline was administered.

Mice subjected to ACL injury (“ACL-I”) were found to exhibit significantly higher OA severity (“OARSI”) scores, higher Piezo1 expression in articular chondrocytes, increased joint innervated nerves with increased CGRP⁺ pain biomarker, a greater area of chondrocyte death induced by mechanical impact, and greater joint-loading on their injured limbs, compared to uninjured mice. Surprisingly, ACL-I mice to which GsMTx4 was administered post-injury exhibited reduced Piezo1 in articular chondrocytes, decreased area of chondrocyte death by mechanical impact, decreased nerve innervation in the knee, decreased CGRP⁺ pain nerves, decreased Piezo2 expression in CGRP⁺ pain nerves, and reduced OARSI scores versus saline-treated ACL-I mice. These results reveal that there is positive correlation between enhanced Piezo1 expression in chondrocytes and enhanced CGRP⁺/Piezo2⁺ innervated pain nerves in osteoarthritic knees and that GsMTx4-treatment post-injury delays OA progression.

Further, the effect of administering GsMTx4 or saline to animals with another type of joint injury was studied. Mice were subjected to a massive rotator cuff tear. Animals were then subcutaneously injected with either GsMTx4 or saline. After 14 weeks, chondrocytes from shoulders subjected to RCT showed increased expression of Piezo1, as well as increased mechano-vulnerability, as measured by chondrocyte cell death, compared to those of the contralateral shoulder subjected only to sham surgery. Cells from shoulders subjected to RCT treated post-injury with GsMTx4, however, had reduced expressed of Piezo1 and the shoulders showed reduced cartilage degeneration, compared to those of animals whose shoulders were not treated post-injury with GsMTx4. Thus, it is believed that injection of GsMTx4 into subjects with rotator cuff injuries shortly after their injury will experience reduced damage to shoulder chondrocytes and consequently, less pain.

### GsMTx4

GsMTx4 is a 34 amino acid peptide first isolated from the venom of a South American tarantula, Grammostola spatulata. The peptide has the sequence GCLEF-WWKCN-PNDDK-CCRPK-LKCSK-LFKLC-NFSF (SEQ ID NO:4), the C-terminal of which is amidated. GsMTx4 inhibits mechanosensitive ion channels. See, e.g., Bowman, et al., Toxicon, 2007, 49 (2): 249-270. doi: 10.1016/j.toxicon.2006.09.030; Gottlieb et al., Current Topics in Membranes. Editor: O. Hamill (Academic Press), 2007, 81-109. doi: 10.1016/s1063-5823 (06) 59004-0. GsMTx4 is commercially available from, for example, Bio-Techne Corp. (Minneapolis, MN, catalog no. 4912) and TargetMoi Chemicals Inc. (Boston, MA, catalog no. TP1670) and has CAS No. 1209500-46-8.

### Targeting GsMTx4 to Cartilage

Various methods of targeting therapeutic agents to the extracellular matrix (“ECM”) of cartilage are known in the art. It is expected that GsMTx4 can be used directly, or readily modified by these art-known modalities to efficiently deliver GsMTx4 to cartilage ECM.

In some embodiments, the method of targeting GsMTx4 to cartilage ECM is a peptide tag. Conveniently, a genetic sequence is designed which, when expressed, results in a fusion peptide comprising (1) the GsMTx4 sequence (SEQ ID NO:4) preceded or followed by (2) the sequence of the peptide targeting the fusion peptide to cartilage.

One such targeting peptide tag is WYRGRL (SEQ ID NO:1) (sometimes referred to herein as the “exemplar cartilage targeting sequence”) which binds to collagen II al. Nanoparticles functionalized with this peptide were found to target articular cartilage up to 72-fold more than like particles functionalized with a scrambled peptide sequence. Rothenfluh, et al., Nature Mater., 7:248-254 (2008). doi.org/10.1038/nmat2116 (“Rothenfluh 2008”). More recently, the WYRGRL (SEQ ID NO:1) peptide was conjugated to dexamethasone and the conjugate was reported to be retained “in the deep zones of cartilage through specific interactions with cartilage-specific collagen type II bundles.” See, Formica et al., J Controlled Release, 295:118-129 (2019); doi.org/10.1016/j.jconrel.2018.12.025 (“Formica 2018”). Formica 2018 further reports that dexamethasone was conjugated to polycationic chitosan which also “led to deep and sustained infiltration of the drug into full thickness cartilage.” Id. Accordingly, GsMTx4 conjugated to polycationic chitosan, preferably with a linker cleavable by ester linkage hydrolysis, such as that taught in Formica 2018, is expected to result in deep and sustained infiltration of GsMTx4 into cartilage.

The peptide of SEQ ID NO: 1 can be modified by adding a short linker (such as 1˜4 glycines) that does not interfere with the peptide's cartilage-targeting function, such as GGWYRGRL (SEQ ID NO:2). Similarly, one or more amino acids can be added to the C-terminal end of the WYRGRL (SEQ ID NO:1) peptide. For example, Ai et al., (Bioengineering & Translational Med, 6 (1), January 2021 e10187; doi.org/10.1002/btm2.10187), recently reported the use of the sequence WYRGRLC (SEQ ID NO: 3) as a targeting sequence for therapeutic nanoparticles, which it reported were then entrapped in the dense extracellular matrix of the cartilage. It is anticipated that other amino acids or two or more amino acids, can be added to the C-terminal end of the WYRGRL (SEQ ID NO: 1) peptide without interfering with the ability of the peptide to target GsMTx4 to cartilage ECM. Any particular peptide in which additional amino acids have been added to either the N-terminal end, to the C-terminal end, or both of WYRGRL (SEQ ID NO:1) can be readily tested to determine if the resulting peptide can continue to target GsMTx4 to cartilage ECM by, for example, testing the resulting peptide in an assay, such as that taught in Rothenfluh 2008. Versions of WYRGRL (SEQ ID NO:1) bearing additional amino acids at the N-terminal end, the C-terminal end, or both, which do not target at least 50% as much GsMTx4 to cartilage as does WYRGRL (SEQ ID NO:1) are not suitable, while versions of the peptide which directs the same or more amount of GsMTx4 to cartilage are preferred.

Other methods of targeting peptides or other therapeutic agents to cartilage are known and can be also used to transport GsMTx4 to cartilage. Cartilage has an anionic charge from its concentration of sulfated glycosaminoglycan-containing proteoglycans and can be passively targeted by materials that are cationic, such as globular proteins and dendrimers. Agents that specifically bind collagen type II, such as anti-collagen type 2-antibodies, can also be used to target cartilage. In 2018, Brown et al. compared the targeting of cartilage using passive targeting cationic nanoparticles (“NPs”) for electrostatic attraction to cartilage, active targeting NPs with binding peptides for collagen type II, and untargeted, neutrally-charged NPs. Brown et al., Acta Biolmaterialia, 101:469-483 (2020); doi.org/10.1016/j.actbio.2019.10.003 (“Brown 2020”). The authors reported, first, that, targeting strategies significantly improved NP associations with both normal and OA-like cartilage and, second, that the targeted NPs accumulated in OA-like cartilage in higher amounts than they did in healthy cartilage.

As another method, the practitioner can take advantage of known interactions between molecules to deliver GsMTx4 to cartilage ECM. For example, GsMTx4 can be fused to an agent, such as streptavidin, that is known to bind strongly to a partner molecule, in this case, biotin. The partner molecule (here, biotin) can then be injected into the cartilage ECM of the joint to be treated and the fusion GsMTx4-strepavidin protein can then be injected into the joint or into the cartilage ECM. Conversely, the GsMTx4 can be biotinylated and the streptavidin can be injected into the joint or into the cartilage ECM, followed by injection of GsMTx4.

As noted, embodiments in which the moiety used to deliver GsMTx4 to cartilage ECM is a peptide are convenient, as a nucleotide sequence can be created and then expressed to produce the targeting peptide-GsMTx4 as a fusion peptide. In some embodiments, the targeting moiety is a polycation, such as polycationic chitosan, which is attached to GsMTx4 by a linker. In other embodiments, the GsMTx4 may be loaded into cationic nanoparticles, such as those taught by Brown 2020, supra. Other compositions that can target GsMTx4 to chitosan are known, as exemplified by Hu et al., Bioconjugate Chem, 26 (3): 383-388 (2015); doi.org/10.1021/bc500557s.

Any particular peptide or other chemical moiety to be used to target GsMTx4 to cartilage can readily be tested for its ability to do so. For example, a peptide to be considered as a candidate for targeting GsMTx4 to cartilage can be tested by expressing the peptide-GsMTx4 fusion protein and GsMTx4 not fused to the peptide, injecting the joints of a first group of mice with the fusion protein and the joints of other mice with GsMTx4, and comparing the infiltration and retention of GsMTx4 in the joints of the two groups of mice.

Finally, as is clear from the above discussion, there are various ways to enhance delivery of GsMTx4 to cartilage. For purposes of this disclosure, any non-toxic fusion peptide, composition conjugated to or complexed to GsMTx4, or nanoparticle loaded with GsMTx4 or having GsMTx4 on its surface (collectively, “GsMTx4 compositions”) that targets the GsMTx4 to cartilage and that does not interfere with the ability of the GsMTx4 to reduce Piezo1 expression in a joint into which the GsMTx4 composition is injected or infused will be referred to herein as a “targeted GsMTx4 composition” or a “GsMTx4 conjugate.”

### Cartilage and Joints

It is believed that the methods and compositions taught herein can be used to reduce inflammation in the cartilage of synovial joints, including those in the knee, hip, shoulder, ankles, and the facet joints of the spine.

Articular cartilage is a connective tissue that covers the osseous components of synovial joints and serves to absorb impact and bear loads. In this disclosure, references to “cartilage” refer to articular cartilage unless otherwise specified, reference is being made to a body part that is not or is not part of a synovial joint, or as otherwise required by context. Cartilage is composed largely of proteoglycans such as chondroitin sulfate proteoglycan 1 (“aggrecan”), enmeshed within a matrix of collagen fibers. See, e.g., Martel-Pelletier et al., Best Practice & Res Clin Rheumatology, 22 (2): 351-384 (2008);

Cartilage is a resilient tissue, yet 25% of Americans lose mobility and suffer from cartilage degeneration in weight-bearing joints. Articular cartilage in knee joints functions as a cushion withstanding cyclic mechanical loads of up to ˜20× an individual's body-weight. Chondrocytes are the cartilage-resident cells maintaining extracellular matrix (“ECM”) homeostasis. Mechanical cues influence chondrocyte biosynthesis which alter ECM metabolic (anabolic and catabolic) balance via mechanotransduction, a conversion process of mechanical stimuli into intracellular biochemical responses. Chondrocytes are intrinsically mechanosensitive, and sense a wide-range of mechanical loading due to abundantly expressed mechanically-activated (“MA”) ion channels, including Piezo1, Piezo2, and TRPV4.

Mechanical stimuli activate MA channels to flux ions rapidly, depolarize the cell membrane, and initiate downstream signaling cascades including changes in gene expression and protein synthesis. When ionic influx involves Ca²⁺, intracellular Ca²⁺ ions function as a second messenger influencing cell fate decisions regarding proliferation, transcription, secretion, and apoptosis. Since chondrocytes experience arrays of mechanical loads, including compression, tension, and osmotic pressure through the ECM, the ECM composition and modulus of ECM influence chondrocyte mechanosensitivity and metabolism. Chondrocytes are embedded in the ECM of the cartilage, which is avascular, and therefore rely on diffusion to receive nutrients and other factors. Delivering a therapeutic agent to chondrocytes is therefore effected by delivering the agent to the cartilage ECM, with the expectation that it will then diffuse through the proteoglycans, collagens, and other constituents of the ECM to the chondrocytes.

### Mechanically-Activated (“MA”) Ion Channels Piezo1 and Piezo2

In 2010, Coste et al., reported the discovery of two genes that encoded proteins, Piezo1 and Piezo2, required for mechanically activated cation conductance. Coste et al., Science, 330 (6000): 55-60 (2010); DOI: 10.1126/science.119327). Piezo1 and Piezo2 are ion channels that sense hyper-physiologic levels of loading. Chondrocytes sense substrate deformation and membrane stretch on culture dishes in vitro high magnitude cyclic tensile strain of 13-18% has been shown to activate Piezo1 and Piezo2 in chondrocytes. It has been shown that Piezo1 and Piezo2, but not TRPV4 channels, sustain the high-level compression-mediated Ca²⁺ influx that underlies the injury response to mechanical stress.

Piezo1 and Piezo2 homologues are expressed in different cell types and function in distinctive manners. For instance, Piezo1 channels are highly expressed in red blood cells, cardiac fibroblasts, lung epithelial cells, smooth muscle cells, and osteoblasts, sensing forces and adjusting cell migration, proliferation, and tissue remodeling. On the other hand, Piezo2 is robustly expressed in mechano/pain-sensory neurons, vagal sensory neurons and Merkel cells, sensing forces involved in limb movement, airway stretch, light touch, and mechanical allodynia pain development. Research regarding the distribution and function of Piezo1 is reviewed in, for example, Lai et al., Biol Rev Camb Philos Soc., 97 (2): 604-614 (2022); doi: 10.1111/brv.12814. Epub 2021 Nov. 15. Research regarding Piezo2 is reviewed in, e.g., Szczot et al., Annu Rev Biochem. 2021 Jun. 20; 90:507-534. Doi: 10.1146/annurev-biochem-081720-023244.

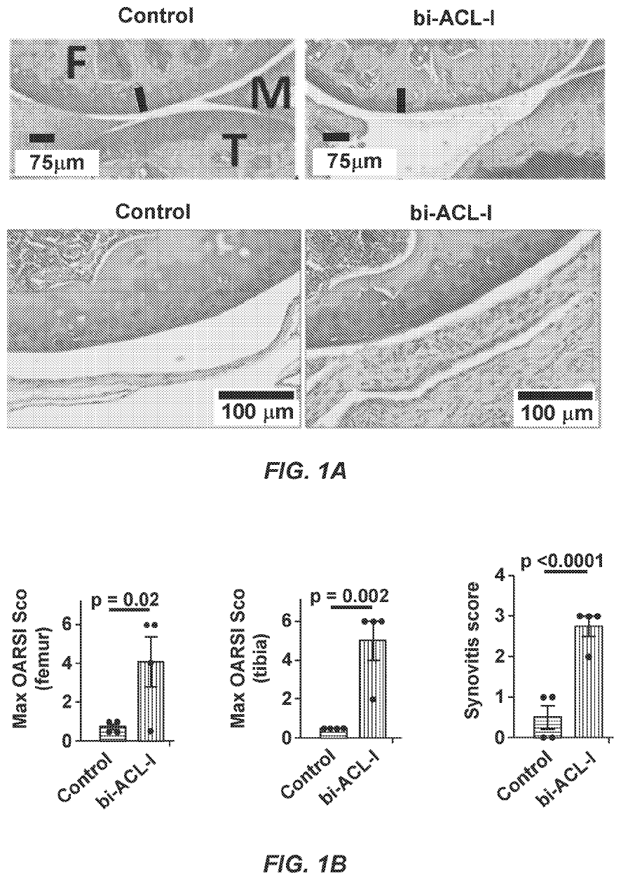

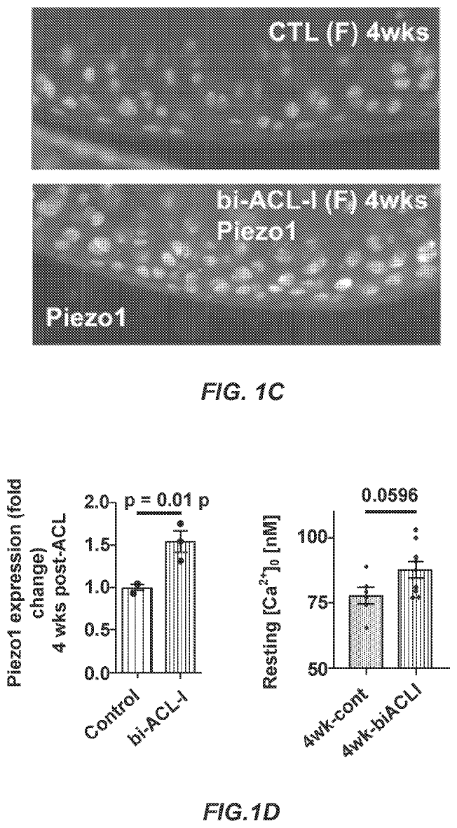

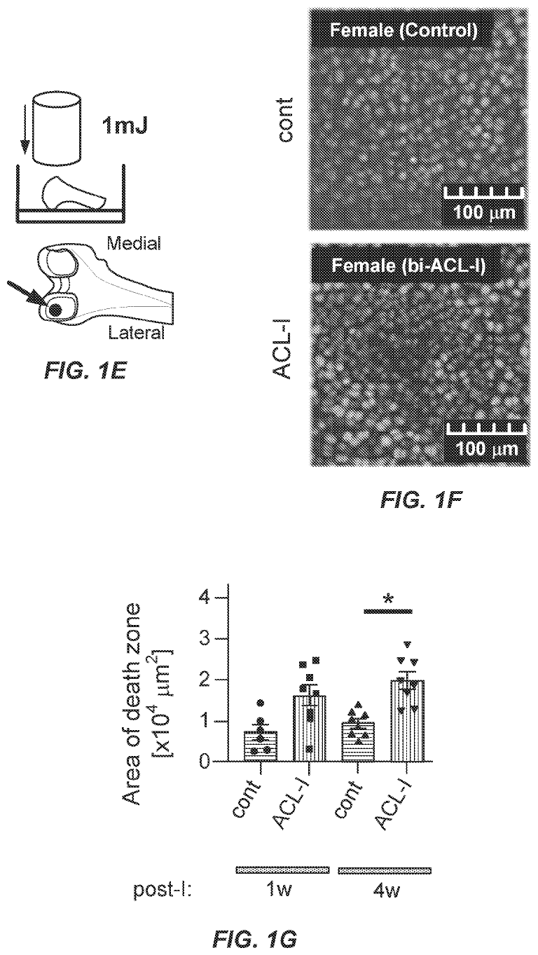

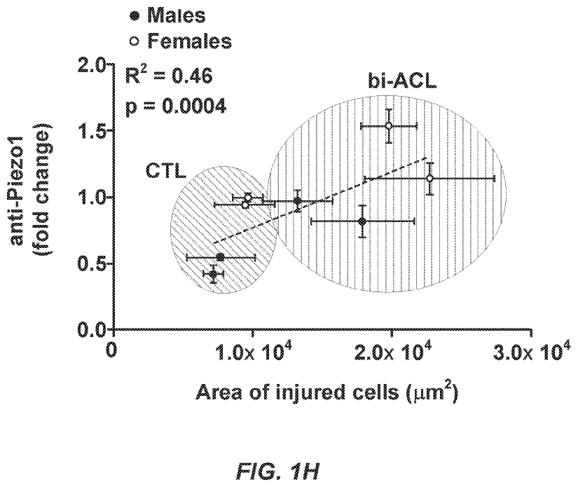

Piezo1 and Piezo2 also have distinct activation and inactivation kinetics that respond to different magnitudes and rates of mechanical stimuli, while gain- or loss-of-function mutations perturb energetics of Piezo1 or Piezo2 ion transport through changes in activation or inactivation times. Piezo1 and Piezo2 have distinct gene expression patterns and gating properties, driving specialized cellular mechanosensitivity. Chondrocytes are a unique post-mitotic cell type that robustly express both Piezo1 and Piezo2. The data shown in the studies reported in the Examples and the Figures show that Piezo1 is upregulated following joint injury. See, FIG. 1C. Without wishing to bound by theory, it is believed that this indicates that Piezo1 has a role in the degeneration of cartilage and the development of OA following injury to a synovial joint.

Studies underlying the present disclosure confirmed the robust expression of Piezo1 and Piezo2 in murine knee joints by immunohistochemistry with knock-out verified antibodies. Both Piezo1 and Piezo2 are highly expressed in superficial zones of articular cartilages of the femur and tibia, as well as in meniscus, but are less expressed in mid- and deep-zones of articular cartilages. Chondrocytes in superficial zones experience higher compressive loading than chondrocytes in deep-zones, suggesting mechanosensitive-adaptation of chondrocytes allowing them to cope with local mechanical environments. Chondrocytes with activated Piezo1 channels are more susceptible to mechanical injury; cartilage pre-treated with Yoda1, the only known Piezo1-specific agonist, exhibit significantly higher chondrocyte vulnerability as compared to vehicle-treated controls in contralateral limbs. Without wishing to be bound by theory, these data support the use of Piezo1 inhibition to promote chondrocyte survival.

In addition to articular cartilage degeneration, other hallmarks of OA include joint inflammation (synovitis) and joint pain. Interestingly, Piezo1 or Piezo2 are also expressed in collateral non-chondrocyte cells that contribute to joint synovitis and pain in OA, so that GsMTx4 treatment is predicted to additionally suppress inflammatory signaling in resident sensory and immune cells. First, synovial macrophages play a critical role in inflammatory responses with an imbalance between pro-inflammatory (M1) and anti-inflammatory (M2) synovial macrophages. Macrophages express high levels of Piezo1, and Piezo1 activation stabilizes HIF1a and upregulates IL1ß inflammatory cytokines. Thus, without wishing to be bound by theory, GsMTx4 may reduce inflammation by inhibiting Piezo1 in macrophages. Second, Piezo2 is dominantly expressed in mechano/pain-sensory neurons and plays a critical role in mechanical allodynia. Pain-sensory neurons innervated in subchondral bone and synovial membrane express Piezo2 channels, and Piezo2-expressing nociceptors have been shown to have a role in early pain behaviors in a meniscus-injury mouse model. Thus, it is expected that GsMTx4 can be used to reduce joint pain or discomfort post-injury.

### Osteoarthritis

Osteoarthritis (“OA”) is the most common form of arthritis and occurs when cartilage within a joint degrades. According to the website of the National Institute on Aging of the U.S. National Institutes of Health, it is one of the most frequent causes of physical disability among older adults. OA is the most prevalent cartilage disorder affecting 32 million individuals in the US, and there will be an estimated 70 million OA patients by 2030. Joint injury is a risk factor for OA.

Among the reasons suspected of causing OA is stress on a joint that has been subjected to an injury. For a knee, for example, that injury may be a tear of one or more of the four main ligaments in the knee: the anterior cruciate ligament (“ACL”), the posterior cruciate ligament (“PCL”), the medial collateral ligament (“MCL”), and the lateral collateral ligament (“LCL”). Anterior cruciate ligament-injury (“ACL-I”) is the most common joint injury, with more than 200,000 reconstruction surgeries performed annually in the US. More than 50% of ACL-I patients, regardless of having ACL-reconstruction surgeries or not, develop post-traumatic (“PT”)-OA within 5˜15 years post-injury, and young adults with ACL injuries are more likely to develop OA before they reach age 40.

The Effect of GsMTx4 on Cartilage Damage and Pain after Joint Injury

Some of the studies reported below involved a murine model of animals which were subjected to trauma by injuring an ACL (as an exemplar of injury to any of the four knee ligaments). Without wishing to be bound by theory, it is believed that injury to the ACL changes the mechanical loading on the joint, promoting imbalanced metabolism in articular cartilage, and promoting the development of post-traumatic OA, or “PTOA.” Animals were divided into four cohorts: the first group, the controls, were not subjected to trauma, the second group underwent bilateral ACL injury, but no further treatment, the third group underwent bilateral ACL injury, and was then administered saline solution at days 3, 10 and 17 post-injury, and the fourth group underwent bilateral ACL injury, and was then administered GsMTx4 peptide at days 3, 10 and 17 post-injury. The animals were evaluated by gait analysis, a cell vulnerability assay, and a histological evaluation/immunofluorescence assay. In one aspect, the assays compared the expression of Piezo1 and Piezo2 in chondrocytes from injured joints, from uninjured controls, and from injured joints injured and then treated with GsMTx4 and from joints that were injured and then treated only with saline solution. The study also looked at calcitonin gene-related peptide (“CGRP”)-containing (“CGRP⁺”) nerve endings. CGRP⁺ nerves have been identified as principal coordinators of thermal and mechanical sensitivity in various pain models. See, e.g., Powell, et al., Nat Commun 12, 5812 (2021); doi.org/10.1038/s41467-021-26100-6.

As reported in the Examples and shown in the Figures, bilateral ACL injury increased Piezo1 expression and the resting Ca²⁺ levels in chondrocytes and the joints exhibited a severe OA phenotype in terms of cartilage degradation and synovial inflammation. Synovitis, inflammation of the synovial membrane, is marked by an increase of cells in the membrane and is painful, particularly when the joint is moved. Animals treated post-injury with GsMTx4, however, showed reduced OA degrees, reduced impact-induced mechanical susceptibility, and reduced expression of Piezo1 compared to animals treated after the injury with saline solution. Injured knees also exhibited an increase in CGRP+ nerve endings, but animals treated with the GsMTx4 peptide showed lower levels of CGRP present than did animals treated with saline. The administration of GsMTx4 post-injury also reduced the increase of pain nerves in the meniscus of the injured joints, reduced changes of gait in the animals treated with the peptide, and reduced cartilage degeneration, compared to mice that underwent bilateral ACL injury, but either no treatment or treatment with saline.

Based on the studies reported herein, GsMTx4 can be used as a treatment after injury to a knee and, by extension, to other synovial joints that have undergone an injury, to reduce death of cartilage chondrocytes and to improve functional recovery of the injured joint. By reducing synovitis, administration of GsMTx4 is also expected to result in the subject experiencing less pain when the joint is moved. Further, by reducing the extent of the injury and cartilage death, it is believed that GsMTx4 treatment reduces the potential that the injured joint will develop OA in the future. The therapeutic effect seen in the studies reported herein is believed to be due to the action of GsMTx4.

### The Rotator Cuff and Rotator Cuff Tear-Related Degeneration of the Glenohumeral Joint

The rotator cuff is a group of four tendons from corresponding scapular muscles that coalesce onto the humerus around the shallow ball-and-socket glenohumeral joint to provide dynamic joint stabilization and a wide range of upper extremity motion. Symptomatic rotator cuff disease is common and arises from either acute traumatic injury or chronic degeneration. The incidence of rotator cuff disease increases with age due to chronic tendon degeneration, leading to partial- or full-thickness tears. Over time, full-thickness tears are at risk of enlarging to a massive rotator cuff tear (>5 cm or involving 2 or more tendons), and the cuff no longer provides dynamic stabilization of the glenohumeral joint. The altered biomechanics lead to a distinct degenerative phenotype—cuff tear arthropathy (CTA)—characterized by eccentric articular cartilage wear and bony remodeling of the glenohumeral joint. There are distinct spatial differences in articular cartilage morphology of the humeral head in human specimens retrieved from arthroplasty and in the glenohumeral joint from a mouse model of CTA. In humans, the superior aspect of the humeral head exhibits tearing, fibrillation, and thinning of articular cartilage; whereas, articular cartilage at the center of the humeral head exhibits overall thickening, a multi-layered tidemark zone, and clustered chondrocytes. In the mouse, articular cartilage is also thickened in the central humeral head at early time points following a massive rotator cuff tear, which is hypothesized to correspond to a period of inflammation and remodeling in the altered mechanical environment.

Articular cartilage of the glenohumeral joint resides in a complex and dynamic mechanical environment. Chondrocytes are intrinsically mechanosensitive across a wide range of mechanical loading due to the expression of mechanically activated (MA) ion channels. As noted in preceding sections, Piezo1 is a calcium-permeating channel activated by mechanical stretch at the cell membrane. The critical role of Piezo1 channels in calcium-mediated responses of chondrocytes against injurious loading and inflammatory cues in vitro has previously been reported. Studies in porcine and human knee articular cartilage showed that Piezo1 is primarily activated and regulates calcium flux during both high-strain injurious loading and inflammatory conditions.

Studies reported herein relate to a murine model in which animals were subjected to rotator cuff tears (“RCT”) (surgical ligation of the supraspinatus and infraspinatus tendons) of the right shoulder, with the contralateral shoulder subjected to surgery in which the shoulder was dissected down to the rotator cuff tendons, but the tendons were not ligated (shoulder surgeries in which the tendons are not ligated are sometimes referred to herein as “sham surgeries”). After 14 weeks, chondrocytes from shoulders subjected to RCT showed increased expression of Piezo1, as well as increased mechano-vulnerability, as measured by chondrocyte cell death, compared to those of the contralateral shoulder subjected only to sham surgery. Cells from shoulders subjected to RCT treated post-injury with GsMTx4, however, had reduced expressed of Piezo1 and the shoulders showed reduced cartilage degeneration, compared to those of animals whose shoulders were not treated post-injury with GsMTx4.

Treatment with GsMTx4 or Targeted GsMTx4 Compositions

Treatment the GsMTx4 peptide or targeted GsMTx4 composition is typically performed by administering a therapeutically effective amount locally to damaged cartilage in a joint that has been injured, such as by undergoing a tear of a ligament in the joint. Typically, the administration is by injection. Due to the small size of knee and shoulder joints in mice, the administration of GsMTx4 to those joints in some of the animal studies discussed herein was given by subcutaneous injection.

It is expected that, in humans, injections of GsMTx4 peptide or targeted GsMTx4 composition will be injected directly into a cartilage defect with a syringe, gel pipette or the like. In some embodiments, the injection may be made arthroscopically. Alternatively, the peptide or targeted GsMTx4 composition may be injected after exposing the affected area by a surgical technique such as arthrotomy. It is noted that orthopedists and other doctors routinely treat OA and knee pain by injecting the knee with a variety of therapeutic agents, including corticosteroids, hyaluronic acid, and platelet-rich plasma. Similarly, injection of steroids and other agents into different portions of the shoulder joint have been practiced by orthopedists and other doctors for decades. For example, in 2003, Tallia and Cardone published guidance for family physicians on injecting steroids into the glenohumeral joint from an anterior, posterior, or superior approach, into the acromioclavicular joint, and into the subacromial space. Tallia and Cardone, Am Fam Physician. 67 (6): 1271-1278 (2003). It is therefore expected that practitioners are well familiar with how to provide such injections.

A “therapeutically effective amount” of GsMTx4 peptide is an amount that, alone or together with further doses, reduces chondrocyte death and improves function of the affected joint. It is anticipated that practitioners can readily determine amounts of GsMTx4 or targeted GsMTx4 composition suitable for use in the inventive methods in the course of clinical trials using art-standard methods for determining dosages. It is anticipated that the amount of GsMTx4 peptide or targeted GsMTx4 composition injected will be from 1-500 mg, preferably 10-100 mg, and more preferably 20-80 mg for each joint injected.

Administration of the GsMTx4 or targeted GsMTx4 composition preferably is commenced as soon as any swelling from the injury to the joint is resolved. A single injection may be made, or a first dose may thereafter be followed by one, two, three, or four weekly or biweekly injections.

As the persons determining the patient's course of treatment are typically experienced physicians, it is anticipated that they can readily determine how long the injections should continue based on their observations of the patient's symptoms, including their comfort in moving the affected joint or joints.

### EXAMPLES

### Example 1

This Example reports on the design of some of the studies underlying this disclosure.

Experiments were performed in a non-invasive ACL-injury mouse model. Eight to nine week year old C57BL/6 mice were divided into four groups: (1) controls, which did not undergo injury, (2) animals subjected to bilateral ACL injury, (3) animals subjected to bilateral ACL injury, followed by injections of saline solution at 3 days, 10 days, and 17 days post-injury, and (4) animals subjected to bilateral ACL injury, followed by injections of GsMTx4 on the same day at 3 days, 10 days, and 17 days post-injury. Both male and female mice were used in the four groups and results were tracked by gender as well as by group.

Animals in the ACL-injury groups were provided with buprenorphine as an analgesic, placed under anesthesia by isoflurane inhalation, and had the injuries induced with a custom-built strain gauge-instrumented device by applying axial force along the femoral shaft until a force drop was felt and a distinct “pop” sound was heard. ACL injuries were confirmed with an anterior drawer test by laterally positioning and securing the mice, and then anteriorly pulling their tibia with a 0.2 N force, while X-rays were taken to confirm that the procedure had not caused a fracture.

### Example 2

This Example reports on the results of some studies underlying this disclosure.

OA was induced by a bilateral anterior cruciate ligament-injury (ACL-I) in 8-week-old C57BL/6 mice. Piezo1-labeling intensity in articular chondrocytes and nerves in subchondral bone, the OA severity (OARSI score), the mechano-vulnerability of chondrocytes, and gait parameters was compared among four experimental groups-Control (uninjured), ACL-injured, ACL-injured with saline administration, and ACL-injured with GsMT4 treatment—at 3-weeks post-ACL-I.

ACL-I mice showed significantly higher OARSI scores, Piezo1-labeling in chondrocytes and innervated nerves, area of chondrocyte death induced by mechanical impact, and joint-loading on their injured limbs, compared to uninjured mice. ACL-I mice to which GsMTx4 was administered exhibited reduced Piezo1-labeling in chondrocytes and nerves in subchondral bones, decreased area of chondrocyte death by impact, and reduced OARSI score versus saline-treated ACL-I mice. The results reveal that there is a positive correlation between Piezo1 expression in chondrocytes and innervated nerves with OA degree, and that GsMTx4-treatment post-injury reduced OA degree in this animal model.

### Example 3

This Example reports on the results of studies on aspects of the effect on bone and cartilage in injured joints of animals that have undergone bilateral ACL injury compared to the joints of uninjured animals, as controls.

FIG. 1A presents four photos of knee joints, two from control animals and two from animals that have been subjected to bilateral ACL injury (“bi-ACL-I”). For orientation, the bones and meniscus in the upper left photo are labeled as follows: F=femur, T=tibia, and M=meniscus. The bones and meniscus of the other three photos are in the same orientation as those in the upper right, although the tibias in the animals that have undergone bilateral ACL injury have been displaced relative to those of the uninjured controls. Referring to the top two photos, the left hand photo of the joint of the uninjured control animal shows a thick band of cartilage overlaying the bone of both the femur and the tibia (the stained red band in the original photos shows as a medium-dark gray band in the black and white reproductions). In contrast, in the animal with the bilateral ACL injury, the cartilage has been eroded almost completely away from a substantial portion of the tibia, and is notably thinned on the femur. Referring to the bottom two photos, one can see substantial synovial inflammation (synovitis) in the joint subjected to bilateral ACL injury compared to the joint from the uninjured control.

FIG. 1B presents graphs comparing the osteoarthritis severity, or “OARSI” score for the femur and for the tibia, respectively, as will as the synovitis score. The graphs for the femur and for the tibia show that the OARSI score (abbreviated “Sco” in FIG. 1B) is much higher for the bones from joints that have undergone bilateral ACL injury compared to those from controls, as is the synovitis score.

FIG. 1C presents photographs of the expression of Piezo1 on the cartilage on the surface of a femur (“F”) of a mouse whose ACL was not injured (top photo, labeled “CTL”), and of the expression of Piezo1 in the cartilage on the surface of the femur of a mouse four weeks after bilateral ACL injury (“bi-ACL-I”). As can be seen from the photos, the expression of Piezo1 is significantly upregulated in the cartilage of the joint that has been injured.

FIG. 1D, left side, presents a graph quantitating the expression level of Piezo1 in the cartilage of animals four weeks after bilateral ACL injury relative to the corresponding joint of control animals. On the right side, FIG. 1D presents a graph showing the relative resting levels of Ca²⁺ in the joint of animals four weeks after bilateral ACL injury relative to the corresponding joint of control animals.

FIG. 1E is a diagram showing impact loading applied to cartilage over a portion of a mouse bone as part of an assay for studying the resulting area of chondrocyte death, sometimes referred to a “mechano-death” or “mechano-vulnerability” assay. See, Kotelsky, et al., Osteoarthritis and Cartilage Open, 4 (1): 100227 (2022); doi.org/10.1016/j.ocarto. 2021.100227.

FIG. 1F presents photos of a bone of a control mouse subjected to impact loading, and of a bone of a mouse with a bilateral ACL injury subjected to impact loading. Although it is harder to see in the black-and-white reproduction than in the color original, in which the dead cells are red and the living cells are green, the area of dead cells is much larger in the cartilage of the animal with the bilateral ACL injury than in cartilage of the mouse that was not first subjected to bilateral ACL injury. FIG. 1G presents a graph quantifying the areas of cell death seen in control animals and in animals with a bilateral ACL injury one week (“1w”) after the animals who underwent bilateral ACL injury were injured (“post-I”), and four weeks (“4w”) after the animals who underwent bilateral ACL injury were injured.

FIG. 1H is a graph plotting, on the Y axis, the relative amount of Piezo1 in the cartilage of control animals (“CTL”) and animals that have undergone bilateral ACL injury (“bi-ACL”), as shown by binding of an anti-Piezo1 antibody, and, on the X axis, the area of injured (actually, dead) cells following the application of a punch force on the cartilage.

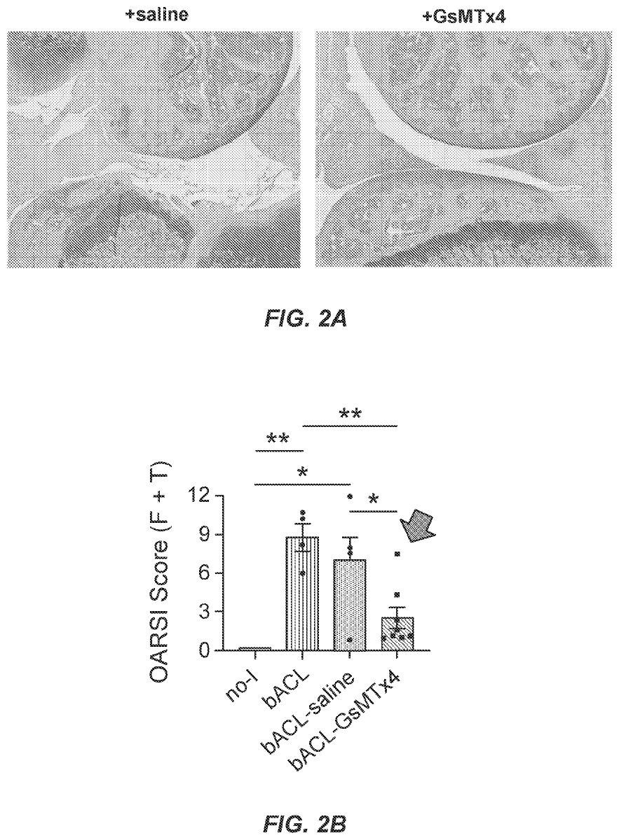

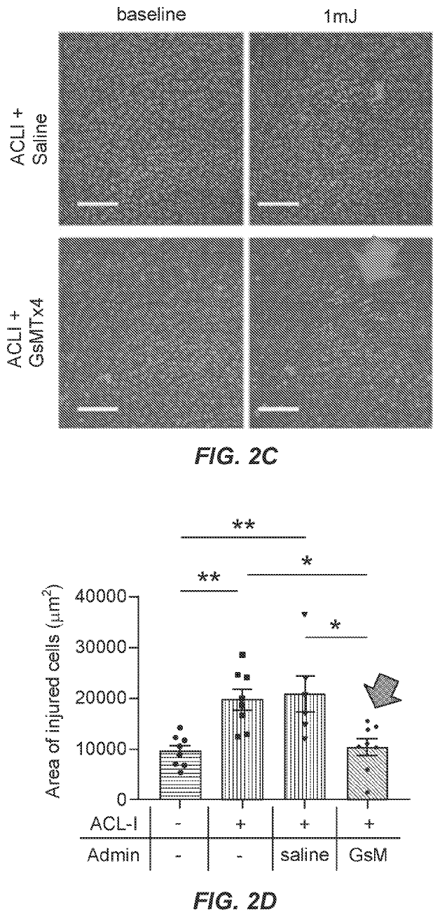

FIG. 2A presents two photos, both from animals that have undergone bilateral ACL injury. The animal in the photo shown on the left was injected after the injury with saline solution (“+saline”), while the one on the right was injected at the same time points post-injury with GsMTx4 (“+GsMTx4”). The cartilage on the bone of the mouse treated with saline is severely eroded and disrupted on the femur and the tibia, while there is still a layer of cartilage almost all of the femur and tibial surface in the animal treated with GsMTx4. FIG. 2B is a graph plotting (on the Y axis) the combined OARSI score for the femur and the tibia of (from left to right across the X axis) animals that were not injured, animals with bilateral ACL injury (“bACL”), animals with bilateral ACL injury, followed by subcutaneous injection of saline solution, and animals with bilateral ACL injury, followed by subcutaneous injection of GsMTx4. As can be seen, animals with bilateral ACL injury, followed by subcutaneous injection of GsMTx4 had a much lower OARSI score than animals that were either not treated or that were treated with saline solution.

FIG. 2C presents two sets of two photos from animals that have undergone bilateral ACL injury. The animal in the photos in the top row was injected after the injury with saline solution (“ACLL+saline”), while the animal in the bottom row was injected at the same time points post-injury with GsMTx4 (“ACLI+GsMTx4”). The left photos show the amount of dead cells (in red, in the color original, compared to living cells, which have taken up a green label) and after application of a 1 mJ force. FIG. 2D presents a graph showing the results of the studies. The Y axis shows the area of dead cells per μm². The X axis shows both whether the animals are uninjured controls (“ACL-I−”) or have undergone bilateral ACL injury (“ACL-I+”), and whether they have been left untreated (“Admin-”) or have been treated with saline or with GsMTx4.

### Example 4

This Example discusses the results of the studies set forth in Examples 1-3.

The results reveal that bilateral ACL injury increases Piezo1 expression and the resting Ca2+ levels in chondrocytes, exacerbate mechanical susceptibility, and result in a severe OA phenotype in terms of cartilage degradation and synovial hyperplasia. GsMTx4 treatment post-ACL injury decreased OA degrees significantly and decreased impact-induced mechanical-susceptibility. Mice administered GsMTx4-D (all the amino acids constituting the peptide in this study were the D enantiomer) by subcutaneous injection post-injury exhibit less articular cartilage loss compared to injured mice without treatment at 3-weeks post-injury. The D enantiomer form of the peptide is expected to be more resistant to proteolytic degradation in vivo and to therefore have a longer residence time in clinical use.

### Example 5

This Example discusses studies in which mice were first divided into three groups: Group 1, control animals, were not injured, but were injected with GsMTx4, Group 2 mice were given a bilateral ACL injury in their hind legs, and then given weekly subcutaneous injections of saline, and (3) Group 3 mice were given a bilateral ACL injury in their hind legs, and then given weekly subcutaneous injections of GsMTx4.

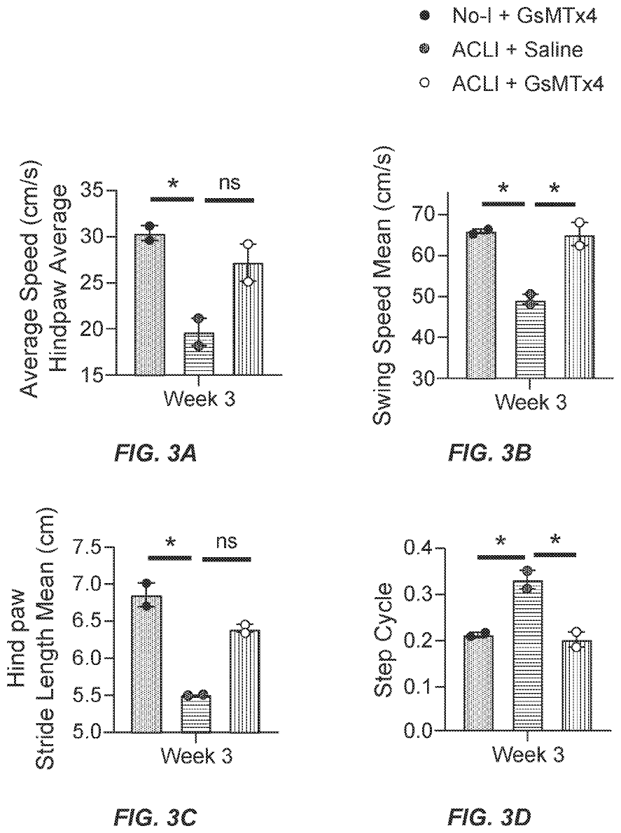

After 3 weeks, the gait of the animals in each group was studied for the following metrics: hind paw stride length, hind paw average speed, hind paw swing speed, and step cycle. As can be seen in the graphs presented in FIG. 3, animals that were given bilateral ACL injuries, followed by GsMTx4 had a gait that was the same or almost the same as that of mice that had not had an ACL injured, while those injected with saline did not.

### Example 6

This Example reports on methods and materials used in a study to determine whether GsMTx4 can rescue chondrocytes in a shoulder following a rotator cuff tear.

Ethical approval was obtained from the appropriate institutional committee Humeri were harvested from wild-type 12-22 week old C57Bl/6 male and female mice. Each specimen was placed in calcium imaging buffer with 2 mM calcium chloride and subsequently incubated with 10 μM calcein-AM for 30 minutes at 37° C. To indicate intracellular esterase activity of living cells, each specimen was incubated with 10 μM yoda1, a Piezo1-specific agonist, 5 nM GSK101, a TRPV4-agonist, or control (DMSO 0.25%, n=7-10/group) in calcium imaging buffer for 15 minutes. Specimens were then incubated with 1.5 nM ethidium homodimer-1 for 3 minutes to indicate loss of plasma membrane integrity in dead cells. The specimen was then arranged in a custom 3-D printed holding device such that the chondrocytes within the apex of sphericity of the humeral head were facing the glass well of a dish to be imaged on an inverted confocal microscope. A baseline confocal image with overlay of 100 μm Z-stacks was obtained with 488 and 594 nm wavelengths to image live and dead cells, respectively. The humerus was then transferred to a custom impact loading apparatus. A 2 mJ load was applied to the lateral aspect of the proximal humerus, such that the convex apex of the humeral head cartilage was impacted against the glass microscope dish. A 2 mJ load was chosen based on preliminary work by Kotelsky to define mechanic-vulnerability of distal femur and proximal humerus articular chondrocytes in the custom loading apparatus. The samples were again incubated in ethidium homodimer-1. A post-injury confocal image with overlay of 100 μm Z-stacks was obtained. A custom MATLAB image processing algorithm was developed to quantify cell viability. The confocal images were processed as maximum intensity projections with threshold via Otsu's method. Then, watershed segmentation, distance transform, and centroid approximation algorithms were created and applied to segment and quantify individual cells based on intensity values. This facilitated the cell counting process, allowed quantification of the total cell area, and allowed adjustment of the region of interest between before- and after-impact micrographs.

A massive rotator cuff tear (RCT) model was performed by surgical ligation of the supraspinatus and infraspinatus tendons of the right shoulder of 14-week-old C57Bl/6 male and female mice through a deltoid splitting approach. Mice were anesthetized with 2% isoflurane gas mixed with oxygen. A single dose of buprenorphine SR (0.5 mg/kg) was injected subcutaneously at the nape. The right shoulder was prepped using a chemical hair removal agent, followed by betadine solution for 2 minutes and a rinse with 70% ethanol. The remaining steps were performed under sterile technique. A #11 scalpel blade was used to sharply incise the skin and subcutaneous tissues in a longitudinal incision over the palpable border of the lateral shoulder. The deltoid fascia and muscle were sharply incised in line with the skin incision with care not to dissect distally along the humerus to protect the axillary nerve. A pair of forceps were then used to grasp the proximal humerus and pull laterally to place the glenohumeral joint on gentle tension. A 25G needle was passed under the supraspinatus and infraspinatus tendons. Dissecting scissors were used to sharply divide the tendons as proximally as possible, and the tendon stump was sharply removed with scissors. The deltoid fascia was then reapproximated with a single 8-0 vicryl suture in a simple stitch. The skin was closed with 5-0 nylon suture in simple stitches. The mice were recovered on a 37° C. warmer prior to being returned to their home cage. Sham surgery was performed on control animals such that a deltoid splitting approach was used to dissect down to, but not ligate, the rotator cuff tendons. The contralateral (left) shoulder served as an uninjured control. Animals were monitored daily for 3 days post-operatively, and no adverse events were noted. The animals were then maintained in a standard unrestricted cage environment for the follow-up duration. Animals were sacrificed 4 weeks post-operatively. An additional cohort of 14-week-old C57Bl/6 male mice underwent the same surgical intervention with sacrifice at 14 weeks post-122 operatively to assess longer term sequelae of massive RCT on the glenohumeral joint articular cartilage. A series of mice who underwent surgical rotator cuff ligation were injected with GsMTx4 in reconstituted sterile saline (n=3) or a sterile saline control (n=3). Injections were administered subcutaneously twice weekly post-operatively. One animal in the GsMTx4 group was sacrificed early due to malnutrition; thus, it was not included in the final analysis.

Humeri were harvested and chondrocyte mechano-vulnerability was quantified before and after 2 mJ impact according to methods above. Forequarter amputation of the fore limbs at the scapula was performed on each mouse bilaterally to keep the glenohumeral joint and associated muscular anatomy intact. Specimens were fixed in 10% neutral buffered formalin for 72 hours prior to decalcification in 14% EDTA “Webb Jee” solution for 7 days. Specimens were then paraffin embedded and sectioned for resultant coronal orientation, with 7 μm sections taken from the center of the glenohumeral joint. Sections were then deparaffinized and rehydrated through a series of xylene and alcohol baths. The central section from each joint was then stained with Safranin O to localize cartilaginous tissue. Average cartilage thickness was measured over the arc of the humeral head from the central histologic section by taking the average of 12 measurements from the articular surface to the tidemark at the superior, middle, and inferior aspects of the humeral head (OlyVIA v3.4.1, Olympus).

For Piezo1 immunohistochemistry, deparaffinized and rehydrated slides were incubated in citrate buffer (10 mM sodium citrate, 0.05% Tween-20, pH 6.0) for 3 hours min at 75° C. Sections were blocked with 5% Normal Goat Serum (NGS) in 1×PBST for one hour and incubated overnight at 4° C. with rabbit anti-mouse primary antibodies to Piezo1 (1:200; Proteintech #15939-1-AP). Sections were washed with PBS prior to incubation for one hour at room temperature with Alexa Fluor 594-conjugated goat anti-rabbit secondary antibody (1:1000; Cell Signaling #8889S) diluted in 5% NGS in 1×PBST. Slides were washed with 1×PBST and 1×PBS, then mounted with ProLong Gold containing 4′,6-diamidino-2-phenylindole (DAPI; Life Technologies). Chondrocyte Piezo1 protein expression was quantified by average threshold intensity of immunofluorescence by ImageJ analysis through raw intensity averaging.

Paired student t-tests were used to evaluate for differences between treatment and contralateral control groups, unpaired student t-tests were used to compare sham with surgical intervention, and ANOVA was used to compare multiple groups, with statistical significance set to p<0.05. A minimum of three biologic replicates were used for each experiment.

### Example 7

This Example reports on the results of a study to determine whether GsMTx4 can rescue chondrocytes in a shoulder following a rotator cuff tear.

Initial experiments were performed on freshly explanted, in situ proximal humeral head articular cartilage to evaluate the baseline mechano-vulnerability of chondrocytes in response to injurious impact loading. For male mice, there was a statistically significant increase in humeral head chondrocyte cell death following impact loading after culture with Yoda1 relative to controls, indicating that Piezo1, in part, drives chondrocyte vulnerability to injurious loading. In contrast, there was a statistically significant decrease in humeral head chondrocyte cell death following impact loading after culture with Gsk101 (a TRPV4-agonist), suggesting a chondroprotective role with TRPV4 activation. Articular chondrocytes from female humeri had higher baseline chondrocyte mechano-vulnerability, such that there was nearly double cell death for injurious loading after incubation with control media. Unlike the male mice, there was not a significant increase in chondrocyte cell death following incubation with Yoda1, nor was incubation with Gsk101 chondroprotective.

Rotator cuff surgical ligation and sham surgery (deltoid splitting approach without RC ligation) was performed on 14-week-old male and female mice. The first cohort of male and female mice were sacrificed at 4 weeks post-operatively. There were no discernable differences in the Safranin O-stained histology of male or female mice that underwent RC ligation versus sham surgery or contralateral controls. Similarly, there was no difference in average cartilage thickness of the humeral head for male or female mice that underwent RC ligation versus sham surgery or contralateral controls. The humeri were explanted from three animals from each group, and these specimens were subjected to impact loading to assess mechano-vulnerability. There was a significant increase in chondrocyte cell death after impact loading for proximal humeri from RCT injured joints versus contralateral uninjured controls. There was a non-significant trend in increased chondrocyte cell death from joints that underwent sham surgery relative to the uninjured contralateral joint in the same animal. There was no difference between surgical intervention, sham, or respective contralateral control joints in quantitative Piezo1 protein immunofluorescence of articular cartilage 4 weeks after intervention for males or females. Based on the results of no significant differences from both the in situ mechano-vulnerability studies and the 4-week post-operative analysis, females were excluded from a second surgical cohort to evaluate the glenohumeral joint 14 weeks after rotator cuff ligation. There were notable differences in the gross appearance of the humeral head from male mice that had undergone rotator cuff ligation relative to contralateral controls. Specifically, there was an area of denuded and dehydrated cartilage with underlying bone bruising at the anterior superior quadrant of the humeral head. Despite these visual differences, there were no significant difference in the humeral head cartilage thickness for RC ligation versus sham surgery or contralateral controls. Humeri were explanted from 5 animals that had undergone RC ligation, and the specimens were subjected to impact loading to again assess mechano-vulnerability. There was a significant increase in chondrocyte cell death in response to injurious loading for proximal humeri from RCT injured joints versus contralateral uninjured controls. There was a significant increase in chondrocyte Piezo1 protein expression, measured by quantitative immunofluorescence, for joints that underwent RC ligation relative to both the contralateral control (p<0.05) and sham surgery (p<0.01).

Surgical ligation of the rotator cuff was performed on six additional animals which were then subjected to systemic subcutaneous injections of saline control or GsMTx4 twice weekly before sacrifice 14 weeks post-operatively. There was no difference in average humeral head cartilage thickness for animals that had undergone RC ligation and received saline versus GsMTx4 or versus the respective contralateral control joint for each treatment. There was, however, a significant increase in humeral head chondrocyte Piezo1 protein expression for animals that underwent RC ligation and received saline injections versus the contralateral control joint (p<0.05) and versus animals that underwent RC ligation and received GsMTx4 injections (p<0.05). Conversely, there was a significantly lower humeral head chondrocyte Piezo1 protein expression for animals that underwent RC ligation and received GsMTx4 injections versus the contralateral control joint (p<0.05).

### Example 8

This Example discusses the results of a study to determine whether GsMTx4 can rescue chondrocytes in a shoulder following a rotator cuff tear.

Mouse humeral head chondrocytes respond to injurious mechanical loading, in part, through the mechanically gated calcium ion channel Piezo1. Activation of Piezo1 with Yoda1 increases the vulnerability of chondrocytes to cell death following impact with injurious loads. Conversely, activation of TRPV4 with Gsk101 attenuates cell death following injurious impact loading, which implies a chondro-protective effect. These results are consistent with prior studies to investigate chondrocyte responses to mechanical loads at both the cellular and tissue level. Specifically, injurious loading and inflammation increases Piezo1 protein expression, intracellular calcium flux, and cartilage catabolism, while physiologic loading increases TRPV4 activation and cartilage anabolism.

A surgically induced massive rotator cuff tear alters glenohumeral joint biomechanical loading, and chondrocytes subsequently increase Piezo1 protein expression and resultant chondrocyte vulnerability to additional injurious loads. Taken together, these results demonstrate that chondrocytes are vulnerable to alterations in the biomechanical loading after RCT. An increase in chondrocyte mechano-vulnerability was significant at both 4 weeks post-operative and 14 weeks post-operative relative to contralateral control limbs; however, an increase in Piezo1 protein expression was only significant at the later time point of 14 weeks. This difference indicates that Piezo1 mechanically gated calcium channels are expressed in a time-dependent manner following injury. Previous work to define the long-term sequelae of a massive RCT in the mouse model reported that the mouse shoulder progresses through histopathological changes toward cuff tear arthropathy similar to that seen in humans. See, Zingman, et al., J Orthopedic Res, 2016; doi.org/10.1002/jor.23383. Zingman et al. reported an early increase in cartilage thickness at 14 weeks during a proposed inflammatory period, followed by progressive loss of humeral head sphericity and subchondral bone architecture; pitting, fibrillation, and thinning of articular cartilage; and superior migration of the humeral head with associated acromial acetabularization. In contrast, the current study found no difference in the thickness of articular cartilage or gross histologic appearance of the glenohumeral joint at 14 weeks post-injury. Of note, the prior study only used female mice and did not assess the biochemical, genetic, or cellular properties of articular cartilage. While there were no differences the overall average humeral head cartilage thickness on histology, an area of bone bruising was observed with overlying unhealthy appearing cartilage at the anterior superior quadrant from acromial abutment. This finding is consistent with a prior study in rats that reported focal defects of superior humeral head cartilage but no difference in overall cartilage thickness 12 weeks for full-thickness RCT compared with uninjured control limbs. That prior study further reported a significant increase in capthesin in humeral head articular cartilage one week following RCT and a trend toward increased matrix metalloproteinases in humeral head cartilage up to 12 weeks post-RCT relative to uninjured contralateral control limbs, indicating an early inflammatory and sustained catabolic response of cartilage in response to a torn rotator cuff.

Rotator cuff pathology in rodent models have previously been shown to share features seen in humans including bursal-sided healing and scar formation following partial rotator cuff tear; progressive tendon retraction, muscle atrophy, and fatty infiltration following complete tendon transection and/or muscle denervation; and impaired gait parameters and shoulder function 6 weeks following massive RCT. Despite being quadrupedal, rodents have shoulder anatomy and function that most closely resembles humans among alternative candidate species, excluding non-human primates. Compared with humans, the rodent coracoacromial arch is of similar morphology and orientation, and the excursion of supraspinatus tendon is contained below the acromial arch with ambulation, burrowing, overhead reaching, and climbing. These anatomical features, along with the histopathological progression toward cuff tear arthropathy make the mouse a suitable candidate species to investigate the response of articular cartilage to a massive rotator cuff tear.

Piezo1 expression resulting from a massive RCT was different between sexes; the significant differences in chondrocyte vulnerability and Piezo1 expression seen in males was absent for females. For baseline studies, females had greater baseline cell vulnerability that was not significantly altered with injurious loading and subsequent Piezo1 or TRPV4 activation. In the rotator cuff injury model, females also exhibited a lower threshold for baseline chondrocyte vulnerability than males but lacked mechano-sensitivity or altered protein expression after injurious loading.

The current study identifies a temporal association between rotator cuff injury and glenohumeral articular cartilage mechano-vulnerability with Piezo1 expression. Animals were given the systemic Piezo1 antagonist GsMTx4 by subcutaneous injection and compared with control animals injected with saline. A reduction in chondrocyte vulnerability was seen in GsMTx4-injected animals compared with both the contralateral control joint and saline-injected animals.

Blockade of Piezo1 with GsMTx4 provides an approach to protect chondrocytes and reduce the resultant catabolic cascade induced by any joint injury, including a massive rotator cuff tear. It is hypothesized that restoration of glenohumeral joint biomechanics with rotator cuff repair would abrogate Piezo1 activity to restore cartilage homeostasis and increase the longevity of the joint.

Overall, this study provides evidence that mouse humeral head chondrocytes respond to injurious mechanical loading through activation of the mechanically gated calcium ion channel Piezo1, and cartilage Piezo1 expression increases following a massive rotator cuff tear. As GsMTx4 inhibits Piezo1 expression, its administration following a rotator cuff tear will reduce pathologic changes to cartilage in the shoulder joint due to the overexpression of Piezo1.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of reducing one or more symptoms from a synovial joint injury in a subject in need thereof, said method comprising administering to said injured synovial joint an effective amount of GsMTx4 or of a targeted GsMTx4 composition that delivers GsMTx4 to cartilage in said synovial joint.

2. The method of claim 1, wherein what is administered to said synovial joint is GsMTx4.

3. The method of claim 1, wherein said targeted GsMTx4 composition that delivers GsMTx4 to said cartilage is a fusion peptide comprising GsMTx4 and a peptide tag comprising WYRGRL (SEQ ID NO:1).

4. The method of claim 3, wherein said peptide tag comprising WYRGRL (SEQ ID NO:1) further comprises a short linker that does not interfere with WYRGRL (SEQ ID NO: 1) cartilage-targeting function.

5. The method of claim 1, wherein said targeted GsMTx4 composition that delivers GsMTx4 to said cartilage is biotin conjugated to said GsMTx4, streptavidin conjugated or fused to said GsMTx4, polycationic chitosan conjugated to said GsMTx4, or an antibody that specifically binds collagen type II conjugated or fused to said GsMTx4.

6. (canceled)

7. The method of claim 1, wherein said administration is by injection into or around cartilage in said synovial joint.

8. The method of claim 1, wherein said reducing of one or more symptoms is a reduction in pain.

9. The method of claim 1, wherein said reducing of one or more symptoms is a reduction in cartilage degeneration.

10. The method of claim 1, wherein said synovial joint injury is to a knee joint.

11-13. (canceled)

14. The method of claim 1, wherein said injured synovial joint is a shoulder joint.

15-16. (canceled)

17. The method of claim 1, wherein said injured synovial joint is a facet joint of a spine.

18. (canceled)

19. A product comprising a composition of GsMTx4 or of a targeted GsMTx4 composition that delivers GsMTx4 of GsMTx4 to cartilage in a synovial joint, for use in reducing one or more symptoms from an injury to said synovial joint.

20. The product of claim 19, wherein said synovial joint having an injury is a knee.

21-22. (canceled)

23. The product of claim 20, wherein said reduction of said one or more symptoms from said injury to said knee is a reduction in impairment of gait consequent to said injury.

24. The product of claim 19, wherein said synovial joint having an injury is a shoulder, a facet joint of a spine, or a hip joint.

25-31. (canceled)

32. The product of claim 19, wherein said reduction of said one or more symptoms from said injury to said synovial joint is pain.

33. The product of claim 19, wherein said product comprises GsMTx4.

34. The product of claim 33, wherein said targeted GsMTx4 composition that delivers GsMTx4 to said cartilage is a fusion peptide comprising GsMTx4 and a peptide tag comprising WYRGRL (SEQ ID NO:1), further comprising a short linker that does not interfere with WYRGRL (SEQ ID NO:1) cartilage-targeting function.

35. The product of claim 19, wherein said targeted GsMTx4 composition that delivers GsMTx4 to said cartilage is a fusion peptide of GsMTx4 and a peptide comprising the sequence WYRGRL (SEQ ID NO:1) is GGWYRGRL (SEQ ID NO:2) or WYRGRLC (SEQ ID NO:3).

36. The product of claim 19, wherein said targeted GsMTx4 composition that delivers GsMTx4 to said cartilage is biotin conjugated to said GsMTx4, streptavidin conjugated or fused to said GsMTx4, polycationic chitosan conjugated to said GsMTx4, or an antibody that specifically binds collagen type II conjugated or fused to said GsMTx4.


SEQUENCE LISTING (4 sequences)

SEQ ID NO: 1 - protein, 6 aa, synthetic construct
WYRGRL

SEQ ID NO: 2 - protein, 8 aa, synthetic construct
GGWYRGRL

SEQ ID NO: 3 - protein, 7 aa, synthetic construct
WYRGRLC

SEQ ID NO: 4 - protein, 34 aa, Grammostola spatulata
GCLEFWWKCNPNDDKCCRPKLKCSKLFKLCNFSF